(19) United States
(12) Patent Application Publication
(10) Pub. No.: US 2014/0377745 A1
(43) Pub. Date: Dec. 25, 2014

(54) Method for Enhanced SNP Discrimination and Detection

(71) Applicants: Rolf Sybren de Kloet, Tallahassee, FL (US); Edwin de Kloet, Tallahassee, FL (US); Siwo Red de Kloet, Tallahassee, FL (US); Arne Herman de Kloet, Tallahassee, FL (US); Caitlin Marie Walsh, Tallahassee, FL (US)

(72) Inventors: Rolf Sybren de Kloet, Tallahassee, FL (US); Edwin de Kloet, Tallahassee, FL (US); Siwo Red de Kloet, Tallahassee, FL (US); Arne Herman de Kloet, Tallahassee, FL (US); Caitlin Marie Walsh, Tallahassee, FL (US)

(21) Appl. No.: 13/921,715

(22) Filed: Jun. 19, 2013

(51) Int. Cl. C12Q 1/68

(52) U.S. Cl. CPC C12Q 1/6858
USPC 435/6.11

(57) ABSTRACT

The present invention describes an enhanced method for detection and discrimination of unique single nucleotide polymorphisms (SNPs) using up to 4 forced nucleotide mismatches near the 3 prime end of an allele-specific primer(s) in a PCR reaction. This method also incorporates a fluorescently labeled primer(s) for detection by capillary electrophoresis. Allele specific primers may also be tailed at the 5 prime end to enable accurate discrimination between different amplicons.

This method can be employed as a singleplex or in a multiplex reaction.

An enhanced method for detection and discrimination of unique single-nucleotide polymorphism (SNP) using up to 4 forced nucleotide mismatches at or near the 3 prime end of a labeled primer(s) and/or tailing the primer(s) at the 5 prime end to enable the accurate discrimination between different amplicons in a single or multiplex reaction using capillary electrophoresis.

1. A method for genotyping and enhanced discrimination and detection of one or more unique single-nucleotide polymorphism (SNP) variants in a biological sample.

2. The method according to claim 1, further comprising whether target variant is homozygous positive or heterozygous or homozygous negative for said target gene variant.

3. The method according to claim 1, wherein said biological sample contains DNA or RNA.

4. The method according to claim 1, wherein the DNA or RNA is from a sample derived from mammal, bird, reptile, fish, crustacean, insect, plant, parasite, bacteria, fungi, and/or virus.

5. The method according to claim 1, wherein the DNA or RNA derived is a biological sample selected from the group consisting of blood, hair, mucosal scrapings, semen, tissue, saliva and cell culture.

6. The method according to claim 3, wherein said DNA or RNA is elongated.

7. The method according to claim 6, wherein said DNA or RNA elongation reaction is a PCR reaction.

8. The method according to claim 7, wherein said DNA or RNA elongation reaction is a primer extension reaction.

9. The method according to claim 7, wherein said DNA or RNA elongation reaction is a primer extension reaction that relies solely on allele specific oligonucleotide primers.

10. The method according to claim 9, wherein each reaction is comprised of a pair of primers immediately flanking the SNP variant site, in which each primer with the 3′ base matching the SNP variants. The reaction also contains a shared primer downstream of the paired allele-specific primers for amplification of the amplicon.

11. The method according to claim 10, wherein each allele-specific primers contains a unique forced mismatch at the second, third, and/or fourth base on the 3′ end of the primer, wherein each allele-specific primer contains a unique mismatch at a different base on the primer sequence.

12. The method according to claim 11, wherein the resulting amplicon differs by at least two to three bases per SNP variant.

13. The method according to claim 11, wherein said nucleotide sequence of the amplicon is produced by said PCR reaction.

14. The method according to claim 10, wherein the pair of non-labeled primers are a tailed-primers using non-complementary sequences at the 5 prime end.

15. The method according to claim 10, wherein said oligonucleotide primer(s) comprise a detectable label or labels.

16. The method according to claim 10, wherein said oligonucleotide primers differ in size and/or detectable label.

17. The method of claim 10, wherein the amplicon is measured by a detectable fluorescent label.

18. The method according to claim 17, wherein said concentration of the amplicon is detected in terms of luminescence intensity.

19. The method according to claim 17, wherein said efficiency is examined by measuring the concentration of the amplified produced by said PCR reaction with an electrophoretic method.

20. The method according to claim 17, wherein the target variants in the amplicon can be distinguished as being homozygous negative, heterozygous, or homozygous positive for said target variant by measuring the size and detectable label(s) of the amplicon with an electrophoretic method.
